(19) United States
(12) Patent Application Publication
(10) Pub. No.: US 2026/0211886 A1
(43) Pub. Date: Jul. 23, 2026

(54) SYSTEMS AND METHODS FOR USING ARTIFICIAL INTELLIGENCE MODELS TO MAINTAIN AND UPDATE PROVIDER DATABASES FOR RECOMMENDING PROVIDERS TO USERS

(71) Applicant: Aetna Inc., Hartford, CT (US)

(72) Inventors: Victor A. Danilchenko, Hartford, CT (US); Karen Martinez, Hartford, CT (US); Swati Nanda, Hartford, CT (US); Tianna M. Wrona, Hartford, CT (US)

(21) Appl. No.: 19/029,260

(22) Filed: Jan. 17, 2025

(51) Int. Cl. G06F 16/2457; G06F 16/334; G16H 10/60

(52) U.S. Cl. CPC G06F 16/24578; G06F 16/3347; G16H 10/60

(57) ABSTRACT

In some instances, a method is provided. The method comprises: collecting data for a plurality of clinical providers; performing data vectorization for the plurality of clinical providers using one or more machine learning-artificial intelligence (ML-AI) models to map the collected data into vectorized data representations within a feature space; obtaining, from a user device, a request for a clinical provider recommendation; processing the user information using the one or more ML-AI models to generate a user vector; comparing the user vector with the vectorized data representations using cosine similarity to determine a subset of the plurality of clinical providers to recommend to the user; generating a list of recommended providers that is responsive to the request from the user device based on the subset of the plurality of clinical providers; and providing, to the user device, the list of recommended providers.

### BACKGROUND

A user may seek to choose a new provider and may be provided with numerous different options. For instance, a user may have moved to a new geographical location or may have changed insurance policies due to a career change and their current provider might not be covered under the new insurance policy. Thus, the user may seek a new provider such as a new general practitioner (GP) or family physician. However, choosing a new provider may be difficult. For instance, ratings, availability, location, demographics, and/or other information may contribute to making the choice for a new provider a multi-dimensional decision, and some information (e.g., whether the provider is accepting new patients, or whether the provider is still even practicing) may be simply not accurately and/or promptly maintained. For example, based on ratings and a geographical location that is close to the user, the user may select a provider to use as their GP. But, even though the provider's webpage may indicate that they are accepting new patients, the webpage might not be up-to-date. Thus, when the user calls the provider to set-up an initial appointment, the provider may indicate that they are no longer be accepting new patients. Accordingly, to help facilitate the process, there remains a technical need to use artificial intelligence models to maintain and update provider databases that are used for recommending providers to users.

### SUMMARY

In some examples, the present application provides a method and system for using machine learning-artificial intelligence (ML-AI) models and/or algorithms to maintain and update provider databases for recommending providers to users. For example, an enterprise organization may manage a provider database that stores information associated with a plurality of providers (e.g., clinical and/or behavioral providers). The provider database may be used for recommending providers to users. For example, using a software application, users may provide user information to a system (e.g., a provider management and recommendation computing system (PMRCS)), and the system may use one or more first ML-AI models and the provider database to recommend one or more providers to the user. Furthermore, as mentioned above, it may be difficult to keep the provider database up-to-date for numerous different providers. As such, the system may further use one or more second ML-AI models to manage and/or update the provider database to ensure that the provider database is up-to-date. For example, to update the provider database, the system may first retrieve provider data records from the provider databases and generate a queue of providers to be updated. For instance, the system may review the provider data records and determine the providers with data records that are most likely to be inaccurate (e.g., based on using the one or more second ML-AI models and/or other algorithms). Following, the system may generate a queue of providers that ranks the providers based on the predicted inaccuracy of their data records. The system may then obtain updated provider data of providers within the queue of providers, and update the provider database based on the updated provider data. As such, the system may continuously update the provider data within the provider database such that the data within the provider database is up-to-date. Then, using the up-to-date provider database, the system may determine recommendations of providers, and provide the recommendations to the user. This will be explained in further detail below.

In one aspect, a method is provided. The method comprises: collecting data for a plurality of clinical providers from a plurality of different data sources; performing data vectorization for the plurality of clinical providers using one or more machine learning-artificial intelligence (ML-AI) models to map the collected data into vectorized data representations within a feature space; obtaining, from a user device, a request for a clinical provider recommendation, wherein the request comprises user information associated with the user; processing the user information using the one or more ML-AI models to generate a user vector; comparing the user vector with the vectorized data representations using cosine similarity to determine a subset of the plurality of clinical providers to recommend to the user; generating a list of recommended providers that is responsive to the request from the user device based on the subset of the plurality of clinical providers; and providing, to the user device, the list of recommended providers.

Examples may include one of the following features, or any combination thereof. For instance, in some examples, collecting the data for the plurality of clinical providers comprises: obtaining provider data from a plurality of clinical provider systems; and obtaining patient review data from one or more patient review data sources.

In some instances, performing the data vectorization for a first clinical provider of the plurality of clinical providers comprises: generating a plurality of first vectorized data representations for the first clinical provider based on processing first collected data, from the collected data, associated with the first clinical provider using the one or more ML-AI models; and mapping a first clinical provider vector for the first clinical provider into the feature space based on generating the first clinical provider vector using the plurality of first vectorized data representations.

In some variations, the one or more ML-AI models comprises a set of encoders, and wherein generating the plurality of first vectorized data representations comprises: separating the first collected data associated with the first clinical providers into a plurality of subsets of the first collected data; and processing each of the subsets of the first collected data using an encoder from the set of encoders to generate a first vectorized data representation from the plurality of first vectorized data representations.

In some examples, a first subset of the plurality of subsets of the first collected data indicates a time duration since a last update of a data attribute for the first clinical provider, and wherein the first vectorized data representation is based on the time duration since the last update of the data attribute for the first clinical provider.

In some instances, the user information comprises user characteristics associated with the user and provider characteristics associated with desired clinical providers.

In some variations, comparing the user vector with the vectorized data representations using the cosine similarity to determine the subset of the plurality of clinical providers to recommend to the user is based on using a number threshold indicating a number of clinical providers that are within the subset of the plurality of clinical providers.

In some examples, performing the data vectorization for the plurality of clinical providers comprises: generating a plurality of vectors for the plurality of clinical providers within the feature space, wherein each of the plurality of vectors is associated with a clinical provider from the plurality of clinical providers and comprises one or more of the vectorized data representations; and storing the plurality of vectors within a provider database, wherein determining the subset of the plurality of clinical providers to recommend to the user comprises comparing the user vector with the plurality of vectors stored within the provider database using the cosine similarity.

In some instances, the method further comprises: obtaining an ideal vector for updating the provider database; populating a queue for updating the provider database based on comparing the ideal vector to one or more vectors from the provider database; and updating the provider database based on the queue.

In some examples, updating the provider database based on the queue comprises: determining a first clinical provider from the queue; obtaining new provider data for the first clinical provider based on using a voice artificial intelligence (AI) agent, one or more application programming interface (API) calls to a provider system associated with the first clinical provider, or performing web scraping; generating new vectorized data representations for the first clinical provider based on the new provider data; and updating a first vector for the first clinical provider within the provider database based on the new vectorized data representations.

In some variations, the collected data comprises patient review data and provider data, and wherein perform the data vectorization for the plurality of clinical providers comprises: using feature engineering to extract sentiments from the patient review data; generating first vectorized data representations within the feature space based on using the one or more ML-AI models to process the provider data; and generating second vectorized data representations within the feature space based on the extracted sentiments from the patient review data.

In some instances, generating the second vectorized data representations within the feature space comprises: generating text embeddings for the extracted sentiments; performing cluster analysis for the text embeddings to generate the second vectorized data representations.

In some examples, the method further comprises: obtaining training data comprising data quality training data and provider quality training data; and prior to performing the data vectorization, training the one or more ML-AI models using the training data.

In another aspect, a system comprising one or more processors and a non-transitory computer-readable medium having processor-executable instructions stored thereon is provided. The processor-executable instructions, when executed by the one or more processors, facilitate: collecting data for a plurality of clinical providers from a plurality of different data sources; performing data vectorization for the plurality of clinical providers using one or more machine learning-artificial intelligence (ML-AI) models to map the collected data into vectorized data representations within a feature space; obtaining, from a user device, a request for a clinical provider recommendation, wherein the request comprises user information associated with the user; processing the user information using the one or more ML-AI models to generate a user vector; comparing the user vector with the vectorized data representations using cosine similarity to determine a subset of the plurality of clinical providers to recommend to the user; generating a list of recommended providers that is responsive to the request from the user device based on the subset of the plurality of clinical providers; and providing, to the user device, the list of recommended providers.

Examples may include one of the following features, or any combination thereof. For instance, in some examples, collecting the data for the plurality of clinical providers comprises: obtaining provider data from a plurality of clinical provider systems; and obtaining patient review data from one or more patient review data sources.

In some instances, performing the data vectorization for a first clinical provider of the plurality of clinical providers comprises: generating a plurality of first vectorized data representations for the first clinical provider based on processing first collected data, from the collected data, associated with the first clinical provider using the one or more ML-AI models; and mapping a first clinical provider vector for the first clinical provider into the feature space based on generating the first clinical provider vector using the plurality of first vectorized data representations.

In some variations, the one or more ML-AI models comprises a set of encoders, and wherein generating the plurality of first vectorized data representations comprises: separating the first collected data associated with the first clinical providers into a plurality of subsets of the first collected data; and processing each of the subsets of the first collected data using an encoder from the set of encoders to generate a first vectorized data representation from the plurality of first vectorized data representations.

In some examples, a first subset of the plurality of subsets of the first collected data indicates a time duration since a last update of a data attribute for the first clinical provider, and wherein the first vectorized data representation is based on the time duration since the last update of the data attribute for the first clinical provider.

In some instances, the user information comprises user characteristics associated with the user and provider characteristics associated with desired clinical providers.

In yet another aspect, a non-transitory computer-readable medium having processor-executable instructions stored thereon is provided. The processor-executable instructions, when executed, facilitate: collecting data for a plurality of clinical providers from a plurality of different data sources; performing data vectorization for the plurality of clinical providers using one or more machine learning-artificial intelligence (ML-AI) models to map the collected data into vectorized data representations within a feature space; obtaining, from a user device, a request for a clinical provider recommendation, wherein the request comprises user information associated with the user; processing the user information using the one or more ML-AI models to generate a user vector; comparing the user vector with the vectorized data representations using cosine similarity to determine a subset of the plurality of clinical providers to recommend to the user; generating a list of recommended providers that is responsive to the request from the user device based on the subset of the plurality of clinical providers; and providing, to the user device, the list of recommended providers.

All examples and features mentioned herein may be combined in any technically possible way.

### BRIEF DESCRIPTION OF THE DRAWINGS

The subject technology will be described in even greater detail below based on the exemplary figures, but is not limited to the examples. All features described and/or illustrated herein can be used alone or combined in different combinations. The features and advantages of various examples will become apparent by reading the following detailed description with reference to the attached drawings which illustrate the following:

FIG. 1 is a simplified block diagram depicting an exemplary computing environment in accordance with one or more examples of the present application.

FIG. 2 is a simplified block diagram of one or more devices or systems within the exemplary environment of FIG. 1.

FIG. 3 is a simplified block diagram depicting an exemplary provider management and recommendation computing system in accordance with one or more examples of the present application.

FIG. 4 is a simplified block diagram depicting an exemplary process being performed by the provider validation system in accordance with one or more examples of the present application.

FIGS. 5A and 5B show an exemplary overview of maintaining and updating provider databases for recommending providers to users in accordance with one or more examples of the present application.

FIGS. 5C and 5D show an exemplary data model for maintaining and updating provider databases for recommending providers to users in accordance with one or more examples of the present application.

FIG. 6 is an exemplary participant environment for maintaining and updating provider databases for recommending providers to user in accordance with one or more examples of the present application.

FIG. 7 is an exemplary process for maintaining and updating provider databases and providing recommended providers to users in accordance with one or more examples of the present application.

FIG. 8 is an exemplary process for providing recommended providers to users using vectorized data representations in accordance with one or more examples of the present application.

### DETAILED DESCRIPTION

Examples of the presented application will now be described more fully hereinafter with reference to the accompanying FIGs., in which some, but not all, examples of the application are shown. Indeed, the application may be exemplified in different forms and should not be construed as limited to the examples set forth herein; rather, these examples are provided so that the application will satisfy applicable legal requirements. Where possible, any terms expressed in the singular form herein are meant to also include the plural form and vice versa, unless explicitly stated otherwise. Also, as used herein, the term “a” and/or “an” shall mean “one or more” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on”.

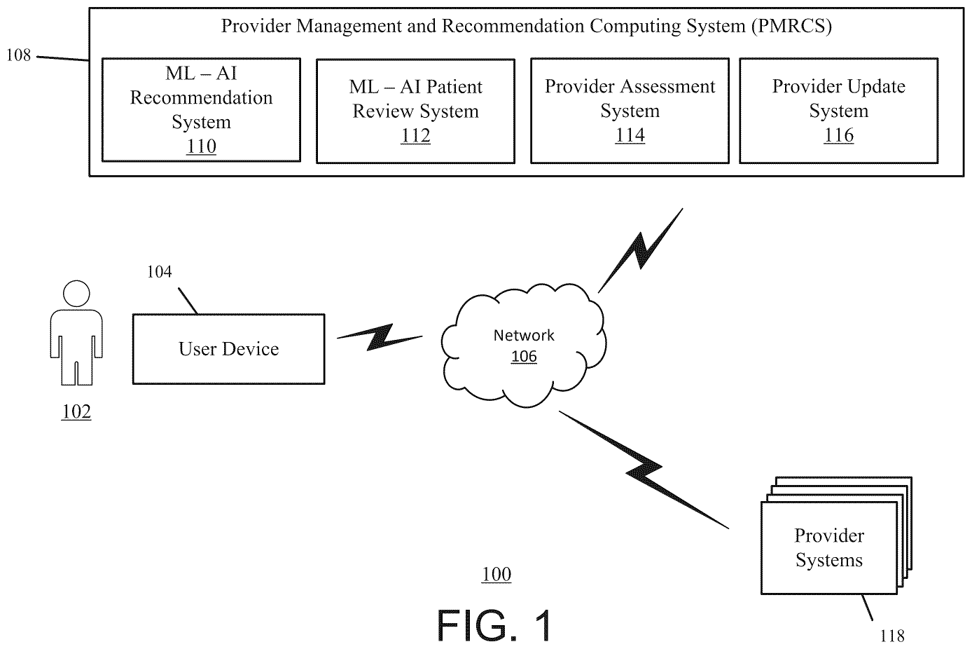

Systems, methods, and computer program products are herein disclosed that maintain and update provider database(s), and use the provider database(s) to provide recommended providers to users. FIG. 1 is a simplified block diagram depicting an exemplary environment 100 in accordance with an example of the present application. The environment 100 includes a user 102, a user device 104, a network 106, a provider management and recommendation computing system (PMRCS) 108, and provider systems 118. The PMRCS 108 includes a machine learning-artificial intelligence (ML-AI) recommendation system 110, an ML-AI patient review system 112, a provider assessment system 114, and a provider update system 116. Although the entities within environment 100 may be described below and/or depicted in the FIGs. as being singular entities, it will be appreciated that the entities and functionalities discussed herein may be implemented by and/or include one or more entities. For instance, the PMRCS 108 may include a plurality of computing devices, systems, platforms, repositories, and/or servers that are spread across multiple different geographical locations and communicate with each other using direct connections and/or the network 106.

The entities within the environment 100 such as the user device 104, the PMRCS 108, and the provider systems 118 may be in communication with other devices and/or systems within the environment 100 via the network 106. The network 106 may be a global area network (GAN) such as the Internet, a wide area network (WAN), a local area network (LAN), or any other type of network or combination of networks. The network 106 may provide a wireline, wireless, or a combination of wireline and wireless communication between the entities within the environment 100

The user 102 may operate, own, and/or otherwise be associated with the user device 104. For example, the user 102 may use the user device 104 to request a recommendation for a new provider. For instance, the user 102 may have moved from one geographical location to another geographical location. Thus, the user 102 may need to switch from a first provider to a second provider. For example, the user 102 may seek a new general practitioner (GP) given that they moved to a new city, and may therefore seek a recommendation for the new GP. In another instance, the user 102 may have switched insurance policies. For instance, the user 102 may have entered employment with a new company that provides the user 102 with a new insurance policy. Thus, the user 102 may seek a recommendation for a new GP that is covered by the new insurance policy. Using the user device 104, the user 102 may provide a request to the PMRCS 108, and the request may indicate user information (e.g., user characteristics) associated with the user 102 such as, but not limited to, the name of the user 102, the insurance policy of the user 102, the geographical location of the user 102 (e.g., domicile of the user 102), provider preferences of the user 102 (e.g., gender of a practitioner associated with the provider) and/or other information. Using the user information, the PMRCS 108 may provide a list of recommended providers to the user device 104, and the user device 104 may display the list of recommended providers.

The user device 104 is and/or includes, but is not limited to, a desktop, laptop, tablet, mobile device (e.g., smartphone device, or other mobile device), smart watch, an internet of things (IOT) device, or any other type of computing device that generally comprises one or more communication components, one or more processing components, and one or more memory components. The user device 104 may be able to execute software applications and/or programs. Additionally, and/or alternatively, the user device 104 may be configured to operate a web browser to connect to a web page and/or applications.

For example, to request a list of recommended providers, the user device 104 may communicate with the PMRCS 108. The PMRCS 108 may be owned, operated, managed, and/or otherwise associated with an enterprise organization. The enterprise organization may be any type of corporation, company, organization, and/or other institution that provides one or more goods and/or services. For instance, the enterprise organization may provide a service such as a recommendation service that recommends providers for users including the user 102. To provide the service, the enterprise organization may manage, operate, and/or host a software application and/or a web page. The user device 104 may access the web page and/or use the software application to communicate with the PMRCS 108. Based on the communications, the PMRCS 108 may determine recommended providers for the user 102, and provide the list of recommended providers to the user device 104.

The PMRCS 108 is a computing system that is configured to manage a provider database, and provide recommendations of providers to the user device 104. For example, in response to receiving a request from the user device 104, the PMRCS 108 may determine a list of recommended providers based on provider data from the provider database, and provide the list of recommended providers to the user device 104. Furthermore, the PMRCS 108 may manage and/or update provider data within the provider database. For instance, provider data may change over time. For example, a provider may reach capacity and may be unable to take on new patients. However, the provider database might not be aware of this change, and may still recommend the provider to users. As such, the PMRCS 108 may use one or more algorithms and/or processes to determine provider data that may be out-of-date and/or inaccurate. The PMRCS 108 may generate a queue of providers (e.g., a list of providers) that are to be updated. Following, using the queue, the PMRCS 108 may use one or more algorithms and/or processes to obtain updated provider data, and update the provider database using the updated provider data.

The PMRCS 108 may include four systems—an ML-AI recommendation system 110, an ML-AI patient review system 112, a provider assessment system 114, and a provider update system 116. The systems 110-116 may be used to manage and/or update the provider database, and use the updated provider database to determine recommended providers for users, including the user 102. Each of the four systems 110-116 may include and/or may be implemented using one or more computing devices, computing platforms, cloud computing platforms, systems, servers, databases, repositories, and/or other apparatuses. In some examples, one or more of the systems 110-116 and/or the PMRCS 108 may be implemented as engines, software functions, and/or applications. In other words, the functionalities of the one or more of the systems 110-116 and/or the PMRCS 108 may be implemented as software instructions stored in storage (e.g., memory) and executed by one or more processors. The functionality of the PMRCS 108 and the systems 110-116 will be described in further detail below.

In some instances, the PMRCS 108 with the four systems 110-116 shown in FIG. 1 is merely an example. In other instances, the PMRCS 108 may include additional systems with additional functionalities and/or capabilities. Additionally, and/or alternatively, the PMRCS 108 might not include each of the four systems 110-116 shown in FIG. 1. For instance, in some variations, the PMRCS 108 may include one system that performs the functionalities of one or more systems shown in FIG. 1 (e.g., the ML-AI recommendation system 110 may also perform the functionalities of the ML-AI patient review system 112).

Each of the provider systems 118 may be associated with a provider that provides medical and/or behavioral services to users (e.g., patients) including the user 102. For example, the provider systems 118 may be associated with a variety of clinical providers, practitioners, institutions, and/or facilities such as medical providers, treatment providers, and/or other types of clinical providers. For instance, the user 102 may seek a medical and/or treatment provider such as a GP and/or a specialist (e.g., an oncologist). Additionally, and/or alternatively, the provider systems 118 may be associated with behavioral health providers. For instance, the user 102 may seek a behavioral health provider that provides treatment for substance misuse, substance abuse, and/or treatment for a mental health condition.

The provider systems 118 may be and/or include, but are not limited to, a desktop, laptop, tablet, mobile device (e.g., smartphone device, or other mobile device), an internet of things (IOT) device, server, repository, database, and/or any other type of computing system that generally comprises one or more communication components, one or more processing components, and one or more memory components. The provider systems 118 may be able to execute software applications managed by, in communication with, and/or otherwise associated with the enterprise organization. Additionally, and/or alternatively, the provider systems 118 may be configured to perform other functions.

In some examples, the provider systems 118 may be and/or include network elements such as servers, databases, and/or repositories that store information associated with the providers. For instance, the provider systems 118 may include a database that stores information about the provider such as whether the provider is accepting new patients. For example, the PMRCS 108 may retrieve data (e.g., provider data) from the provider systems 118 and store the retrieved data within its provider database. Additionally, and/or alternatively, the provider systems 118 may host, manage, and/or operate a web page and/or software application. The web page and/or software application may provide information to the user device 104 and/or the PMRCS 108.

Additionally, and/or alternatively, the provider systems 118 may be and/or include a device and/or system within a provider facility (e.g., a doctor's office). For instance, the provider systems 118 may include a device (e.g., a telephone), and the PMRCS 108 (e.g., using an artificial intelligence (AI) voice feature) may provide an automated call to the device. In some instances, the PMRCS 108 may provide a voice over internet protocol (VOIP) and/or other automated call to the device. The PMRCS 108 may gather information from the provider systems 118 based on the automated call, and update the provider data based on the gathered information.

It will be appreciated that the exemplary environment 100 depicted in FIG. 1 is merely an example, and that the principles discussed herein may also be applicable to other situations—for example, including other types of institutions, organizations, devices, systems, and network configurations. For example, in some variations, the functionalities of the PMRCS 108 and/or the systems 110-116 may be separated into multiple different entities that communicate with each other using the network 106.

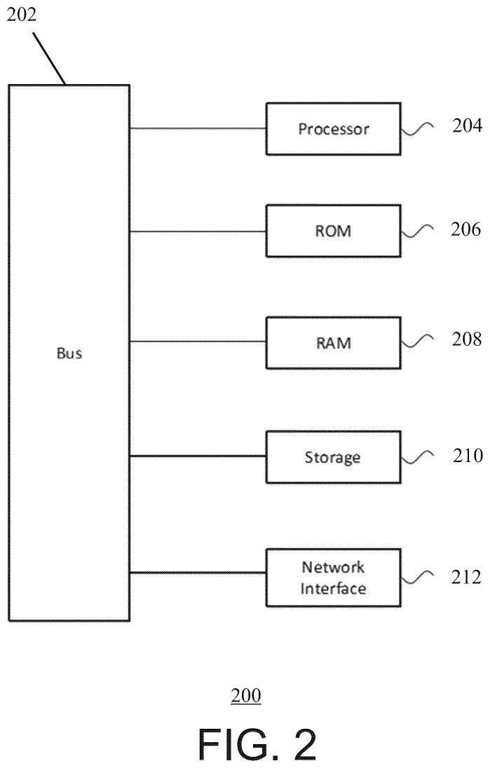

FIG. 2 is a block diagram of an exemplary system and/or device 200 within the environment 100. The device/system 200 includes one or more processors 204, such as one or more CPUs, controller, and/or logic, that executes computer executable instructions for performing the functions, processes, and/or methods described herein. In some examples, the computer executable instructions are locally stored and accessed from a non-transitory computer readable medium, such as storage 210, which may be a hard drive or flash drive. Read Only Memory (ROM) 206 includes computer executable instructions for initializing the processor 204, while the random-access memory (RAM) 208 is the main memory for loading and processing instructions executed by the processor 204. The network interface 212 may connect to a wired network or cellular network and to a local area network or wide area network, such as the network 106. The device/system 200 may also include a bus 202 that connects the processor 204, ROM 206, RAM 208, storage 210, and/or the network interface 212. The components within the device/system 200 may use the bus 202 to communicate with each other. The components within the device/system 200 are merely exemplary and might not be inclusive of every component, server, device, computing platform, and/or computing apparatus within the device/system 200. Additionally, and/or alternatively, the device/system 200 may further include components that might not be included within every entity of environment 100.

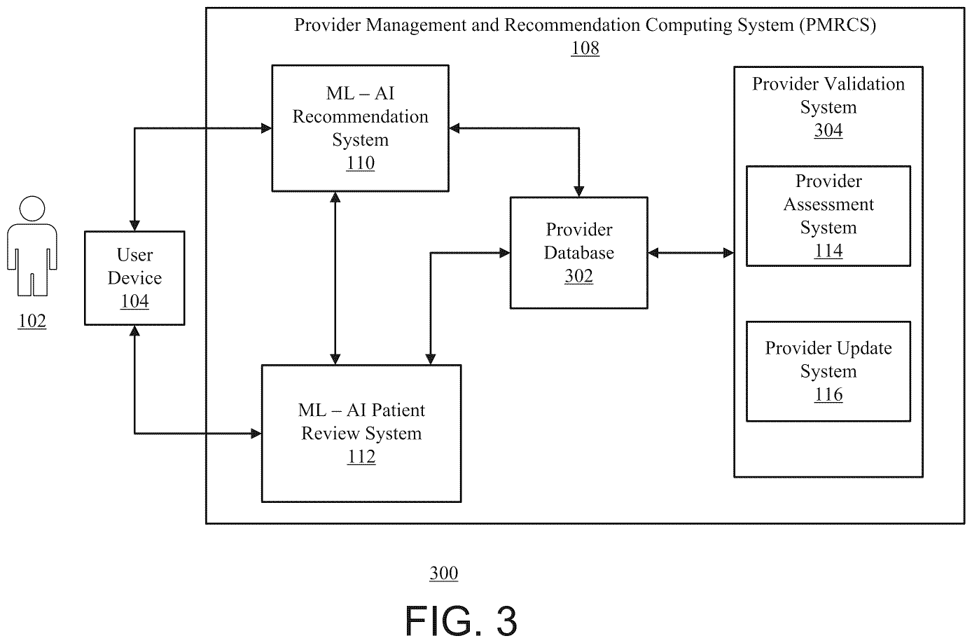

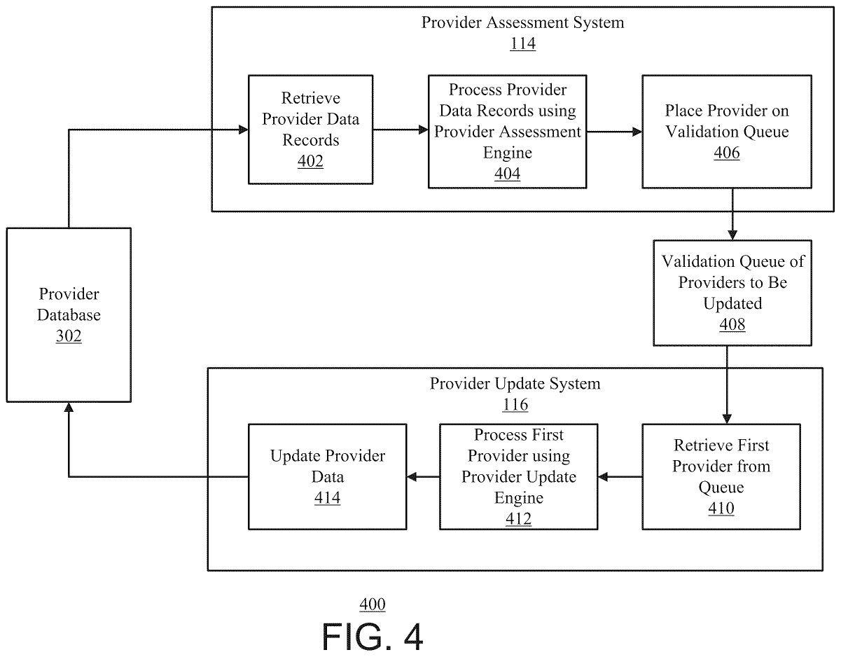

FIG. 3 is a simplified block diagram 300 depicting an exemplary provider management and recommendation computing system 108 in accordance with one or more examples of the present application. For instance, the block diagram 300 includes the user 102, the user device 104, and the PMRCS 108. The PMRCS 108 includes the systems 110-116 that are shown in FIG. 1 as well as a provider database 302 that stores provider data (e.g., provider records) associated with a plurality of different providers (e.g., medical and/or behavioral providers). In addition, the provider assessment system 114 and the provider update system 116 are included within a provider validation system 304, which will be described in further detail in FIG. 4 below.

In operation, the user 102 may use the user device 104 to communicate with the PMRCS 108 (e.g., the ML-AI recommendation system 110 and/or the ML-AI patient review system 112). The PMRCS 108 may be and/or include one or more systems (e.g., systems 110-116) that are configured to extract and/or process provider data (e.g., data related to dental, vision, and/or other medical-related and/or behavioral-related fields) using one or more ML-AI models, and to use the extracted data to provide AI-powered provider recommendations to users searching for a new provider (e.g., medical services provider).

For instance, the user device 104 may provide a request for a recommended provider to the PMRCS 108. Using the ML-AI recommendation system 110 and the provider database 302, the PMRCS 108 may provide one or more recommendations for providers for the user 102 (e.g., a list of recommended providers). The list of recommended providers may indicate a list of practitioners, facilities, a group of practitioners, and/or other medical/behavioral entities. For instance, the ML-AI recommendation system 110 may generate recommendations for a specific user (e.g., the user 102) based on user characteristics and/or provider characteristics. The user characteristics may be characteristics associated with the user 102 such as, but not limited to, the demographics of the user 102, the geographical location of the user 102, the medical history of the user 102, personal preferences of the user 102, and/or other user characteristics. For instance, using a recommendation application, which is described in further detail below, the user 102 may provide user input to the user device 104 indicating user characteristics and/or preferences (e.g., provider characteristics), and the user device 104 may provide the user characteristics and/or preferences to the PMRCS 108. The provider characteristics may be any characteristic (e.g., desired characteristic) associated with a provider such as, but not limited to, provider data and/or provider reviews/sentiments from the ML-AI patient review system 112.

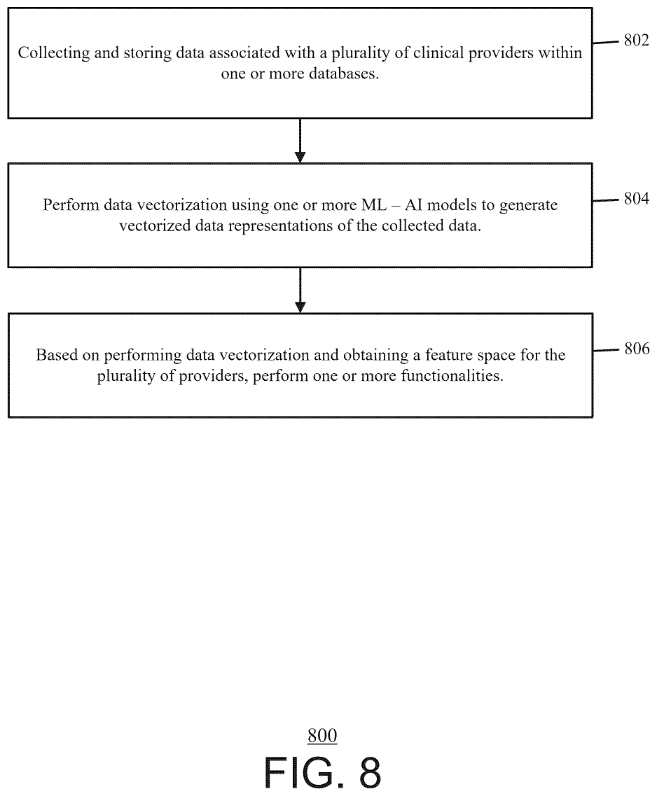

The ML-AI recommendation system 110 may receive the user characteristics and/or provider characteristics, and then employ and/or utilize one or more ML-AI models and/or algorithms to generate the recommended providers for the user 102. For instance, based on processing the characteristics and/or the provider characteristics using the one or more ML-AI models and/or algorithms, the ML-AI recommendation system 110 may generate one or more provider recommendations such as a list of recommended providers. In some examples, the ML
AI recommendation system 110 may use pre-computed provider vectorized data representations to generate the provider recommendations. This is described in further detail in FIG. 8.

In some variations, the user device 104 may communicate with the PMRCS 108, including the ML-AI recommendation system 110, using a software application such as a recommendation application. For example, the user device 104 may execute the recommendation application, and the recommendation application may cause display of one or more display screens to the user 102. For instance, based on executing the recommendation application, the user device 104 may display one or more fields for the user 102 to provide their user characteristics (e.g., user information). Based on user input such as a preference of a gender of a practitioner, the user device 104 may provide the user characteristics to the PMRCS 108. Additionally, and/or alternatively, after receiving the list of recommended providers, the user device 104 may display the list of recommended providers that were generated using the ML-AI recommendation system 110.

The user device 104 and/or the ML-AI recommendation system 110 may communicate with the ML-AI patient review system 112. For instance, the ML-AI patient review system 112 may review and extract sentiments that are then provided to the ML-AI recommendation system 110. The extracted sentiments (e.g., data) may include, but are not limited to, characteristics such as patient wait times, length of time spent with the medical personnel (e.g., doctor) versus the feeling of being rushed, provider's behavior towards the user, and/or other characteristics or sentiments associated with a user's visit to the doctor. For example, the user device 104, which may be the same user device that provided the initial request for a recommended provider to the ML-AI recommendation system 110 or a different user device, may provide a review about the provider to the ML-AI patient review system 112.

For instance, in some examples, the user device 104 may provide a request to the ML-AI recommendation system 110, and may receive a list of recommended providers. The user 102 may then schedule and attend an appointment for a recommended provider from the list of recommended providers. Afterwards, the user device 104 (e.g., based on executing the recommendation application) may provide a prompt for the user 102 to provide their review and sentiments regarding the appointment with the recommended provider, and the user 102 may provide a review. The user device 104 may provide the review to the ML-AI patient review system 112. The ML-AI patient review system 112 may extract (e.g., using one or more ML-AI models and/or algorithms) the sentiments and generate sentiment data. The ML-AI patient review system 112 may then provide the extracted sentiment data to the ML-AI recommendation system 110 directly and/or to the provider database 302. Then, in a subsequent iteration, the same user device 104 or a different user device may provide another request to the ML-AI recommendation system 110 for recommended providers. The ML-AI recommendation system 110 may use the sentiment data that was provided previously to generate a new list of recommended providers, and provide the new list to the user device that is requesting the provider recommendation. In some instances, the PMRCS 108 may include an enrichment processor that may enrich the sentiment data prior to storing the sentiment data into the provider database 302. For instance, the enrichment processor may enrich the sentiment data such as by including, supplementing, modifying, replacing, and/or removing data from the sentiment data that is provided from the user device 104. Then, the enrichment processor may store the enriched sentiment data into the provider database 302. The enrichment processor will be described in further detail below.

To help facilitate the ML-AI recommendation system 110 in generating the list of recommended providers to the user device 104, the PMRCS 108 may use a provider validation system 304 to validate and/or update the provider data that is stored in the provider database 302. For instance, as mentioned above, the provider data within the provider database 302 may be out of date. For example, initially, the PMRCS 108 may extract provider data from the provider systems 118 and/or other data sources. For instance, the PMRCS 108 may obtain provider data such as indicated availabilities from the provider systems 118. Additionally, and/or alternatively, the provider data that is obtained from the provider systems 118 and/or other data sources (e.g., the ML-AI patient review system 112) may include, but is not limited to, age of last update, patient complaints, provider ratings, temporal pattern of provider ratings, and/or other information. After extracting the provider data, the PMRCS 108 may store the provider data within the provider database 302. However, after a set amount of time (e.g., based on the age of last update), the provider data might not be accurate. For instance, a popular provider may have an influx of patients over a short time span, and may thus not have any further availability to take on new patients. Thus, by using outdated provider data, the ML-AI recommendation system 110 may provide recommended providers that are no longer accepting new patients. As another example, the provider may have a series of negative reviews over a short time span due to one or more circumstances. The user preferences may indicate that the user 102 would like to see a highly rated provider and in response, the ML-AI recommendation system 110 may seek to provide recommended providers that are highly rated. But, due to a provider having a series of negative reviews over a recent time span, by using outdated data, the ML-AI recommendation system 110 may provide recommended providers that have low user ratings. Accordingly, the PMRCS 108 may seek to use the provider validation system 304 to maintain up-to-date provider data that is then used by the ML-AI recommendation system 110 to recommend providers to users such as user 102. The provider validation system 304, including the provider assessment system 114 and the provider update system 116, are described in further detail in FIG. 4.

FIG. 4 is a simplified block diagram 400 depicting an exemplary process being performed by the provider validation system 304 in accordance with one or more examples of the present application. For instance, at block 402, the provider assessment system 114 may retrieve provider data records (e.g., provider data) from the provider database 302. For example, the provider assessment system 114 may retrieve data records for one provider from the provider database 302 (e.g., retrieve provider data for a particular provider from the provider database 302).

At block 404, the provider assessment system 114 may process the data records for the one provider using a provider assessment engine. For example, by processing the data record using the provider assessment engine, the provider assessment system 114 may assess the provider data quality based on the data source, freshness, user feedback, user star ratings, successful appointments made, and so on. Based on the assessment, the provider assessment system 114 may provide a ranking and/or score (e.g., risk and/or data quality score).

At block 406, the provider assessment system 114 may place the provider on a validation queue 408. For example, the validation queue 408 may indicate providers that are to be updated by the provider update system 116.

In other words, in some examples, the provider assessment system 114 may use one or more ML-AI models (e.g., assessment ML-AI models that are part of the provider assessment engine) to identify “bad” provider records based on rules and/or scores, and include the identified providers in a validation queue 408. For instance, the provider assessment system 114 may determine and/or obtain one or more business rules such as records that have not been updated in the last six months or missing information on new patients' acceptance and/or wait period. Additionally, and/or alternatively, the provider assessment system 114 may determine and/or obtain additional information such as age of last update, patient complaints, provider ratings, temporal pattern of provider ratings, and/or other information. The provider assessment system 114 may process the business rules and/or the additional information using one or more assessment ML-AI models to determine a score (e.g., risk and/or data quality score). The provider assessment system 114 may place the provider onto the validation queue 408 based on the score.

For example, the validation queue 408 may indicate a list of providers that are in a ranked order that is based on a score (e.g., a data quality and/or risk score) determined by the provider assessment system 114 (e.g., using the one or more assessment ML-AI models). For instance, the output from the one or more assessment ML-AI models (e.g., a score) may indicate a risk that the provider records (e.g., provider data) for that provider, which are stored in the provider database 302, is stale and/or invalid (e.g., not up-to-date). For instance, the validation queue 408 may include a plurality of entries including a first (e.g., top) entry, one or more intermediate entries, and a last (e.g., bottom) entry. Each entry may be associated with an output from the one or more assessment ML-AI models. In some instances, the first or top entry may be considered a “worst” ranked provider (e.g., a provider that has the highest risk of having stale, invalid, and/or out-of-date provider data) and the bottom or last entry may indicate a provider that has a highest chance of having up-to-date and correct provider data.

The provider assessment system 114 may continuously and/or periodically (e.g., after a certain time period such as each hour) retrieve one or more data records for a provider, process the data records for the provider, and place the provider onto the validation queue 408. For instance, based on the provider having a low score (e.g., low risk), the provider assessment system 114 may place the provider lower within the validation queue 408 (e.g., closer to the bottom or last entry of the validation queue 408). But, based on the provider having a high score (e.g., high risk), the provider assessment system 114 may place the provider towards the top of the validation queue 408 (e.g., closer to the first or top entry of the queue 408). In some instances, based on a newly processed provider having a high enough score (e.g., a score that is greater than the score for the current first entry within the validation queue 408), the provider assessment system 114 may replace the first entry provider with the newly processed provider, and insert the replaced entry provider as the second entry within the validation queue 408.

At block 410, the provider update system 116 may retrieve a provider from the validation queue 408. For example, as mentioned above, the validation queue 408 may include a list of providers that are ranked based on a score (e.g., a data quality and/or risk score that is determined by the provider assessment system 114). The provider update system 116 may retrieve a provider from the validation queue 408 such as the highest ranked provider from the validation queue 408 (e.g., the provider associated with the first or top entry of the validation queue 408).

At block 412, the provider update system 116 may process the first provider using a provider update engine. At block 414, the provider update system 116 may update the provider data within the provider database 302, including the vectorized data representations.

By using the provider update engine, the provider update system 116 may validate and/or update the provider data/records based on scraping the web for comments, incorporating star ratings associated with the provider, checking appointments that have been successfully made, and/or by placing AI voice calls to the provider's office (e.g., placing an AI voice call to a computing device associated with the provider system 118 and the provider).

In some examples, using one or more processes and/or methods (e.g., using one or more update ML-AI models such as a voice AI agent), the provider update system 116 may resolve and gather provider data for providers within the validation queue 408. For instance, the provider update system 116 may use a Bot Caller (e.g., an automated Bot Caller that makes calls to provider offices and gathers information using pre-canned prompts). Additionally, and/or alternatively, the provider update system 116 may perform application programming interface (API) calls to provider directories of other health payors and/or competitors to obtain updated data for updating the provider database 302 (e.g., perform API calls to servers that are within the provider system 118). Additionally, and/or alternatively, the provider update system 116 may perform provider web scraping to gather provider information that is then used to update the provider database 302.

In some variations and as will be described below, some provider records might not be able to be updated using any of the above methods, and may be sent to a dead letter queue for manual operational intervention. For instance, providers that might not be processed by the provider update system 116 may be made available to a group of individuals (e.g., an operations team) for manual validation and/or processing. This group of individuals may include experts (e.g., subject matter experts (SMEs)) that may be able to make calls to provider offices and perform apt research to determine current and high-quality data about providers, which is used to update the provider database 302. Once the data is gathered and the bad provider records are updated, the provider update system 116 may provide the updated data back to the provider database 302 and/or a processor associated with the provider database 302 (e.g., an enrichment processor).

In other words, in some instances, the provider update system 116 may continuously and/or periodically obtain the “worst” provider from the queue 408 (e.g., the highest risk and/or ranked provider). Then, for that provider, the provider update system 116 may validate and/or update the data records/provider data that is stored within the provider database 302. The provider update system 116 may validate the data records/provider data based on using various processes, algorithms, and/or data sources such as, but not limited to, website scraping, provider notifications, patient reviews and complaints, a voice AI agent, and/or other data sources, processes, and/or algorithms.

For instance, in some variations, the provider update system 116 may use a voice AI agent, which may automatically place a call (e.g., a telephone call or VoIP call) to a device at the provider system 118 associated with the provider. For instance, using the voice AI agent, the provider update system 116 may request responses. For example, the requests may include, but are not limited to, “Are you accepting new patients?” and/or “If a new patient comes on board, how soon can you see them?” Additionally, and/or alternatively, the provider update system 116 may notify the provider that the voice AI agent is part of the provider onboarding to insurance or to the PMRCS 108, in order to avoid having the voice AI agent being dismissed as spam. Based on the responses (e.g., verbal, spoken responses from an individual at the provider office) and using the voice AI agent, the provider update system 116 may convert the audio responses into another data format such as text and/or vector data formats (e.g., vectorized data representations). Subsequently, the provider update system 116 may use the converted data to update the data records/provider data for the provider. For instance, initially, the provider data may indicate that the provider is accepting new patients and that they can see them within a week of the user 102 making contact. However, using the voice AI agent, the individual at the provider office may indicate that this has changed, and that the provider is no longer accepting new patients or that the provider is still accepting new patients, but it may take them a month to set up the initial appointment. The provider update system 116 may obtain an audio file comprising the response from the individual, and convert the audio file into another data format such as a text data format. Then, at block 414, the provider update system 116 may update the provider data using the converted audio file that was obtained using the voice AI agent.

In other words, referring to FIGS. 3 and 4, the PMRCS 108 may include four entities—an ML-AI recommendation system 110, an ML-AI patient review system 112, a provider assessment system 114, and a provider update system 116. In operation, the provider assessment system 114 may process provider data from the provider database 302 to determine providers that are most likely to include errors, inaccuracies, and/or be out-of-date. The provider update system 116 may obtain a first provider (e.g., a worst ranked provider) from a validation queue 408, and perform data improvements to update the provider data. The ML-AI recommendation system 110 may customize provider recommendations based on patient needs and preferences. The ML-AI patient review system 112 may perform patient review information extraction. Each of these systems may work in sync and/or in a continuous loop to achieve the final product and remain updated with improved information.

For instance, the provider assessment system 114 may be configured to identify the provider data that is “worst ranked” based on the provider data record that includes information that is the most likely to be inaccurate. The data quality may be measured by weighted features such as source, user feedback, last update, user star rating, successful appointments scheduled, and so on.

The provider update system 116 may consider the “worst ranked” data records from the provider assessment system 114 and run a continuous loop for data improvement to obtain the most accurate information. The data quality may be enhanced in several ways including but not limited to: AI generated phone call to the providers' offices, web scraping, star ratings, and so on.

The ML-AI recommendation system 110 may provide recommendations to the patient (e.g., user 102) based on generating a customized “best ranked” provider list. The rankings may be determined by patient demographic needs and/or preferences—for example, the need for a certain type of specialist, preference for a male or female physician, or accepting new patients—and may also consider soft characteristics derived from patient reviews.

The ML-AI patient review system 112 may complete data extraction derived from the patient reviews. The extracted data may include characteristics such as: patient wait times, length of time spent with the doctor versus the feeling of being rushed, provider's behavior towards the patient, and so on.

Additional data points to be considered by the ML-AI recommendation system 110 and/or the provider assessment system 114 may include, but are not limited to, distance from the patient's home, language spoken by provider, provider demographics, telehealth options, insurances accepted, accepting new patients, and/or wait time for an initial appointment.

In some variations, the PMRCS 108 may allow the user device 104 to initiate a user journey. For instance, the user journey may allow the user 102 to use a communication channel of choice such as web, mobile, contact center, AI voice agent, natural language AI Chatbot/Live chat, and/or other communication channels/methods for searching for providers. Based on the search criteria, review scores, geolocation radius, and/or user preferences (e.g., the gender of a physician), the user device 104 may display provider recommendations to the user 102. In other words, using the recommendation application that is described above, the user device 104 may communicate with the PMRCS 108 through a variety of methods. For instance, the recommendation application may include functionalities and/or capabilities such as AI voice agent and/or a natural language AI Chatbot that facilitates communications between the user 102 and the PMRCS 108. For instance, using the AI voice agent and/or the natural language AI Chatbot, the user device 104 may obtain user characteristics (e.g., user preferences such as gender of a physician), and provide the user characteristics to the PMRCS 108. The PMRCS 108 may generate a list of recommended providers using the ML-AI recommendation system 110 and provide the list to the user device 104. The user device 104 may display the list of recommended providers to the user 102.

In some examples, the PMRCS 108 may provide proactive provider recommendations to the user device 104 and the user 102. For instance, based on user health records, user interactions and/or search history (e.g., inquiries about a specific clinic), and/or call summaries, the PMRCS 108 may determine that the user 102 is seeking recommendations for a new provider. For instance, based on user health records, user interactions and/or search history, the PMRCS 108 may identify a user 102 that may seeking a new provider. The PMRCS 108 may communicate with a user device 104 of the user 102 and provide a prompt asking if the user 102 would like to use the PMRCS 108 to provide a list of recommended providers. Based on a response from the user device 104, the PMRCS 108 may use the ML-AI recommendation system 110 to generate a list of recommended providers and provide the list to the user device 104. The user device 104 may display the list of recommended providers to the user 102.

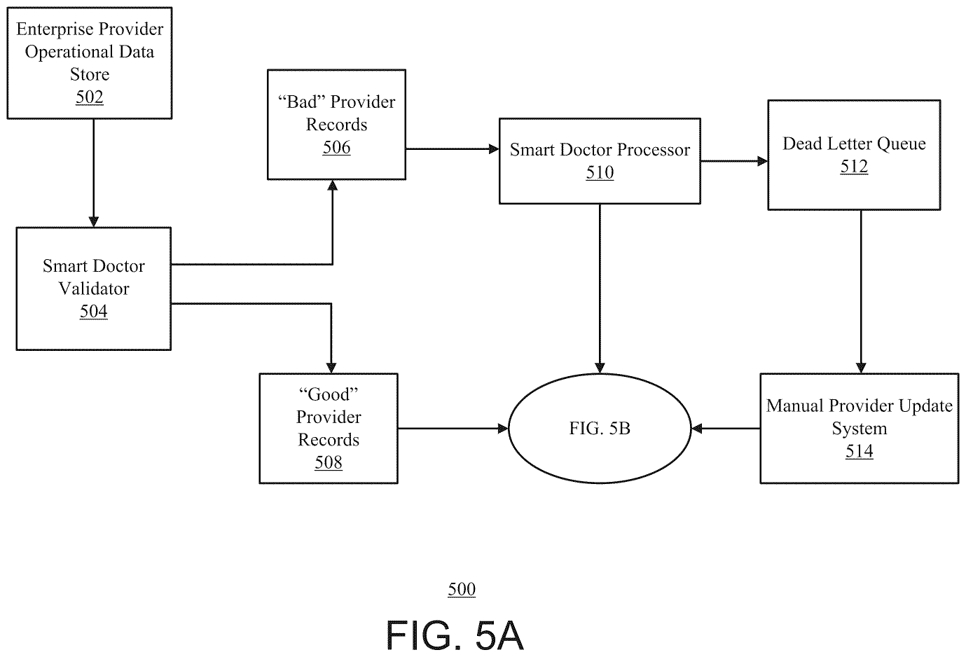

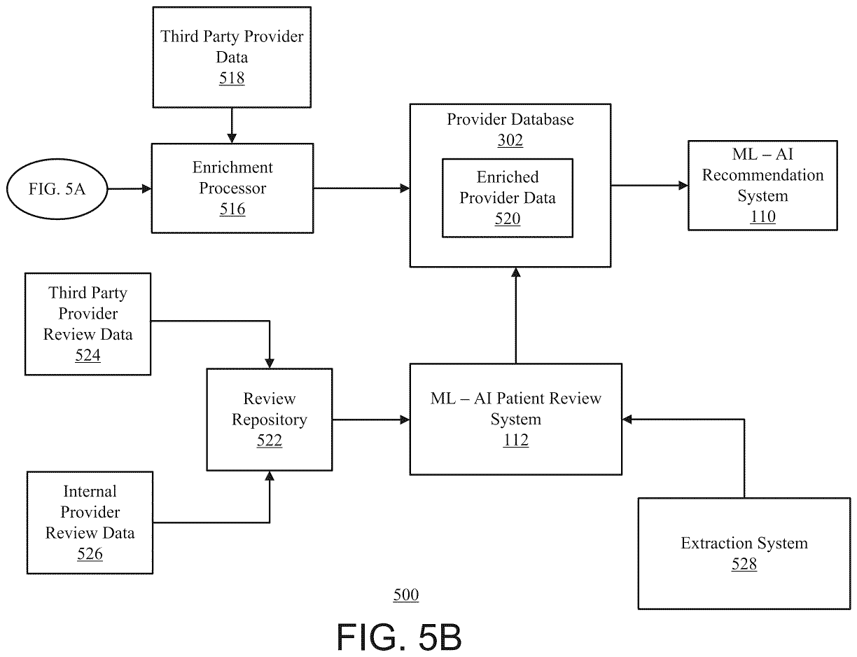

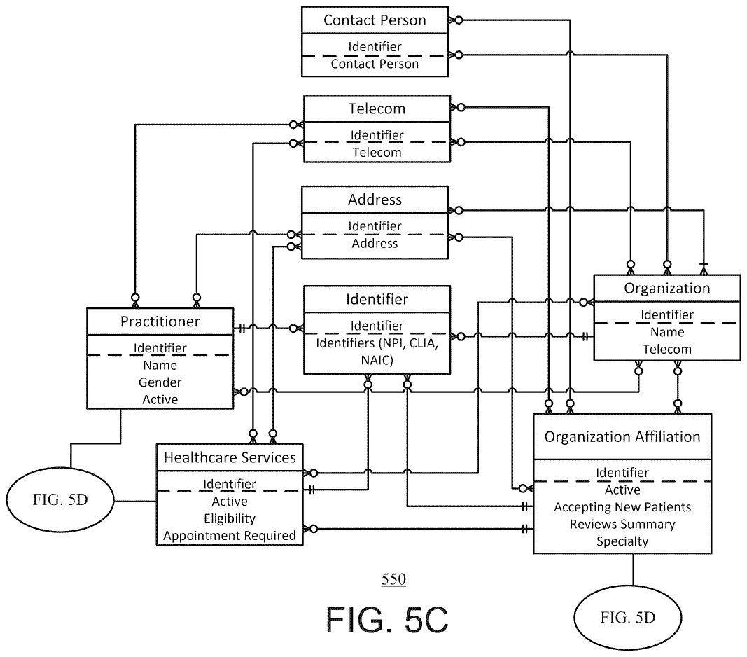

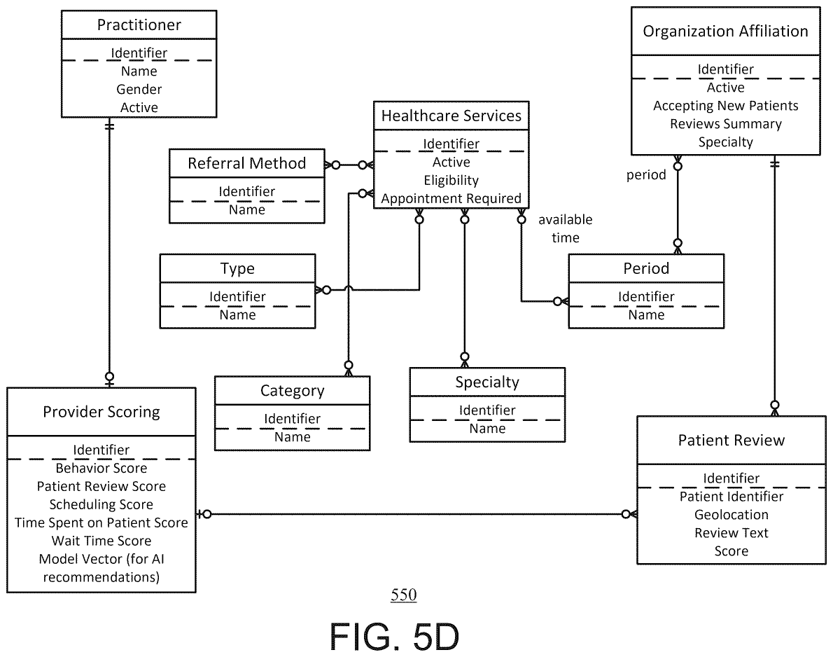

FIGS. 5A and 5B show an exemplary overview of maintaining and updating provider databases for recommending providers to users in accordance with one or more examples of the present application. For instance, the overview 500 may provide a more detailed description of the PMRCS 108 and/or the systems 110-116 described above.

Referring to FIG. 5A, the overview 500 includes an enterprise provider operational data store 502, which may include and/or be one or more databases, repositories, and/or storage elements. The enterprise provider operational data store 502 may be and/or include enterprise data sources that provide provider data. In some instances, the enterprise provider operational data store 502 may include the provider database 302 shown in FIG. 3. In other instances and as shown in FIG. 5B, the provider database 302 may be separate from the enterprise provider operational data store 502. For instance, the provider database 302 may store only enriched provider data 622 that is used by the ML-AI recommendation system 110. However, the enrichment processor 516 may be optional. When not present, the enterprise provider operational data store 502 may include the provider database 302. Initially, aspects of overview 500 will be described with the enrichment processor 516. Afterwards, aspects without the enrichment processor 516 will be described.

The data from the enterprise provider operational data store 502 may be fed (e.g., provided) to the smart doctor validator 504. In some instances, the smart doctor validator 504 may be and/or include the same or similar functionalities as the provider assessment system 114 that is described above. The smart doctor validator 504 may be and/or include a component (e.g., the provider assessment engine) that includes one or more ML-AI models (e.g., assessment ML-AI models). The assessment ML-AI models may be used to identify “good” provider records 508 or “bad” provider records 506 based on rules and scores associated with the records, and then provides the “bad” provider records 506 to the smart doctor processor 510. The determination of the “good” or “bad” provider records 506 is described above. For instance, using the assessment ML-AI models, the smart doctor validator 504 may determine a score (e.g., risk and/or data quality score such as a cosine similarity score that is based on an ideal vector, which is described in further detail in FIG. 8 below). The smart doctor validator 504 may compare the score with one or more thresholds (e.g., a threshold associated with the cosine similarity score). Based on the comparison, the smart doctor validator 504 may determine whether the data record and/or the provider associated with the retrieved data record is a “good” provider record 508 or a “bad” provider record 506.

For example, as mentioned above, the PMRCS 108 and/or the systems 110-116 may utilize a queue of providers (e.g., a validation queue 408) indicating a list of clinical providers to be updated within a provider database. The queue of providers may indicate one or more “bad” provider records 506 and one or more “good” provider records 508, and the “bad” provider records 506 may be updated by the provider update system 116. For example, the queue of providers may indicate a first or top entry, which may be a “worst” or a “bad” provider record 506, and the remaining provider records within the queue of providers may be the “good” provider records 508. The provider update system 116 may then update the “bad” provider record 506 from the queue (e.g., based on using blocks 510-514). In other words, in some variations, the smart doctor validator 504 may generate the queue of providers (e.g., the validation queue 408), which separates the “bad” provider records 506 from the “good” provider records 508 (e.g., based on the ranking within the queue of providers). Following, as mentioned above, blocks 510-514 may describe the provider update system 116 updating the “bad” provider records 506. In addition, the updating of the provider records 506 and 508 may be in a continuous loop such that the “worst” provider records (e.g., the top or first entry within the queue) are consistently updated from the queue, all of the other records are reassessed such that a new “bad” or “worst” provider record is determined, and the new provider record that is on top of the queue is then updated.

The data being provided to the smart doctor validator 504 may include data that is not enriched. For instance, this data may include provider data such as, but not limited to, organization data, practitioner data, organization/affiliation data, patient review data, location data, and/or healthcare service data. The organization data may be associated with an organization (e.g., a grouping of people or organizations with a common purpose such as a group of providers). The practitioner data may be associated with a practitioner (e.g., a person who is directly or indirectly involved in the provisioning of healthcare or related services such as physicians, dentists, pharmacists, and so on). The organization/affiliation data may be associated with data describing the relationship between two distinct organizations. The healthcare service data may be associated with a healthcare service (e.g., a category of services that are provided by an organization at a location such as a pharmacy, psychology services, or a computed tomography (CT) scan with contrast).

The smart doctor processor 510 may obtain the “bad” provider records 506. In some instances, the smart doctor processor 510 may be and/or include the same or similar functionalities as the provider update system 116 that is described above. For instance, the smart doctor validator 504 may store the “bad” provider records 506 into a queue such as the validation queue 408 that is described above. The smart doctor processor 510 may retrieve a provider from the validation queue 408 (e.g., the provider associated with the first or top entry from the validation queue 408). Then, as mentioned above, the smart doctor processor 510 may use one or more processes to update the provider data (e.g., the “bad” provider records 506). For example, the smart doctor processor 510 may be and/or include a component that uses one or more ML-AI models (e.g., update ML-AI models). The smart doctor processor 510 may use one or more methods and/or processes to resolve and/or update the provider data with high quality information using a Bot Caller, API Caller, Third Party Data Comparison, and/or Provider Data Web scraping, which are described above. For instance, the Bot Caller may make a call to the provider office and capture information using pre-canned prompts. The API caller may make a call to APIs exposed by the competitors for provider data (e.g., provider directory APIs). The Third Party Data Comparison may obtain provider data from a third party and correct provider records with relevant data. The Provider Data Web scraping may use web scraping to gather provider data and update the “bad” provider records 506.

In some instances and as described above, the smart doctor processor 510 may be unable to update the provider data. In such instances, the smart doctor processor 510 may place the “bad” provider record 506 in a dead letter queue 512. The manual provider update system 514 may take providers/“bad” provider records 506 that are placed in the dead letter queue 512 and update the “bad” provider records 506. For instance, an individual and/or a team or group of individuals may resolve the provider records that are unable to be resolved by one or more automated processes that are performed by the smart doctor processor 510. For example, using an operational dashboard, human resolution (e.g., call and/or search), and/or data correction, the manual provider update system 514 may update the “bad” provider records 506 based on user input. In some instances, the manual provider update system 514 may be unable or fail to resolve the “bad” provider records 506 and may label the provider associated with the “bad” provider records 506 as invalid.

Subsequently, based on the smart doctor validator 504 indicating that the provider records are “good” provider records 508 and/or based on the smart doctor processor 510 and/or the manual provider update system 514 resolving and/or updating the “bad” provider records 506, the overview 500 proceeds to FIG. 5B and the enrichment processor 516.

Referring to FIG. 5B, the “good” provider records 508 and the updated “bad” provider records 506 are provided to and processed by the enrichment processor 516. For instance, using third party provider data 518, the enrichment processor 516 may enrich the provider records and generate enriched provider data 520, which is then stored in the provider database 302. The enrichment processor 516 may use high quality data (e.g., from the third party provider data 518) to further enrich the provider data, and may store the enriched provider data 520 in the provider database 302.

For example, the enriched provider data 520 may include similar data to the data that was provided to the smart doctor validator 504. For instance, the enriched provider data 520 may include provider data such as, but not limited to, organization data, practitioner data, organization/affiliation data, patient review data, location data, and/or healthcare service data. Additionally, and/or alternatively, the enriched provider data 520 may include, modify, replace, and/or remove data from the data that was provided to the smart doctor validator 504. For instance, the enriched provider data 520 may include provider scoring data that is determined by the enrichment processor 516. The provider scoring data may include, but is not limited to, a provider identifier, a scheduling score, a wait time score, a patient review score, a patient review text, a behavior score, a time spent on patient score, and/or a model vector (e.g., for AI recommendations). For instance, the provider scoring data may indicate which provider data is to be updated first. In some instances, the provider score may reflect or indicate a difference between the original data and the enriched data, and in some variations, the enriched data may have a higher score. To put it another way, the enriched data may be of both higher quality, and may have attributes that are not included in the original data (e.g., an average wait for the first visit, reported bedside manner, and so on).

In other words, the PMRCS 108 may use one or more data models to manage the provider records. For instance, the PMRCS 108 may include syndication and/or proprietary data, which may include provider rosters that are shared by third party providers, provider demographics and/or credentials, and/or data quality metrics. Furthermore, the PMRCS 108 may be associated with a federation path. For instance, information may be shared by and/or with the PMRCS 108 through a common standardized interface such as Fast Healthcare Interoperability Resources (FHIR) and/or an industry wide standard definition for provider information. Furthermore, the PMRCS 108 may use a publish/subscribe model that may require a well-defined cost model for consumption. In some instances, the PMRCS 108 may use one or more types of data models, such as a data model to manage the validation of the provider records by the smart doctor processor 510 and/or the smart doctor validator 504 (e.g., this may represent both organizations such as groups of providers and the individual practitioners that make up that organization). Furthermore, the PMRCS 108 may also use another type of data model such as an enriched data model that associates the validated providers to the data produced from the enrichment process (e.g., enriched provider data). The enriched provider data 520 may be derived through a high-quality enrichment process that uses provider reviews and enterprise sources of provider data (including third-parties). Furthermore, the enriched provider data 520 may be stored within an operational data store (e.g., the provider database 302) and may have a unique key associated back to the individual provider that may be separate and/or distinct from the data provided by a third-party (e.g., the third party provider data 518). The enriched provider data 520 may be applicable to a single practitioner, or it can be applicable to an entire practice/organization (with many providers). The enriched data model, which may be used by the enrichment processor 516, may be flexible to support associating the enriched provider data 520 to a full record as defined by the smart doctor processor 510 and/or the smart doctor validator 504. Alternatively, the enriched data may be associated to a specific provider identifier, and the identifier may be used to associate the enriched data back to the data model used by the smart doctor processor 510 and/or the smart doctor validator 504.

In some instances, the PMRCS 108 may obtain the third party provider data 518 and/or generate the enriched provider data 520 based on a FHIR standard. For instance, a HEALTH LEVEL SEVEN (HL7) FHIR standard defines a method for exchanging healthcare information between different computer systems regardless of how the healthcare information is stored in those systems. For instance, the standard allows healthcare information, including clinical and administrative data, to be available securely to those who have a need to access it, and to those who have the right to do so for the benefit of a patient receiving care. The standards development organization HL7 uses a collaborative approach to develop and upgrade FHIR.

The FHIR API may be a RESTful, or REpresentational State Transfer (REST), approach for data exchange. REST defines categories of data, or “Resources,” to exchange data. The philosophy behind FHIR may be to create a set of Resources that, individually or in combination, satisfy most common use cases. The Patient Resource, for example, includes demographic data related to a patient, such as their name, address, and phone number. Resources also improve granular data retrieval, so that a request returns just the relevant data rather than a full record or document that itself must then be searched.

Once they are modified for specific requirements using FHIR's built-in capabilities, combinations of Resources are brought together in an Implementation Guide to address a specific use case, such as a provider directory, payor-to-payor exchange, provider access to patients' health data, or patient-reported outcomes. This structure itself is flexible for expansion beyond FHIR's core capabilities. FHIR uses modern security standards, authentication and encryption including authorization. Similarly, among FHIR's privacy capabilities, FHIR may support labeling sensitive information so that only those who have the need, and the right can see it. FHIR may enable both the sender and receiver to have the same understanding of health care data.

In some examples, the enriched provider data 520 may include pre-computed feature vectors (e.g., vectorized data representations) in accordance with the provider/patient/demand domain model, such as to facilitate the interactive match between the patient and the provider. This is described in further detail in FIG. 8 below.

Following, as mentioned above, the ML-AI recommendation system 110 may use the provider database 302 and/or the enriched provider data 520 to generate and provide a list of recommended providers to a user device 104. For instance, the ML-AI recommendation system 110 may enable users to search for providers, and use the provider scores to recommend providers based on patient preferences and/or other factors.

Furthermore, the overview 500 includes the ML-AI patient review system 112, which is described above. The ML-AI patient review system 112 may obtain review data from the review repository 522. For instance, third party provider review data 524 and/or internal provider review data 526 may be provided to and stored within the review repository 522. The ML-AI patient review system 112 may process the reviews within the review repository 522 and provide the processed data to the provider database 302. For example, the ML-AI patient review system 112 may perform sentiment classification and/or analysis to score the provider reviews and feed them into the provider database 302 (e.g., incorporate them into the enriched provider data 520).

The extraction system 528 may provide aspects of the enriched provider data 520 to other entities (e.g., third parties). For instance, certain aspects of the enriched provider data 520 may be proprietary, but other aspects may be able to be provided to third parties. As such, the extraction system 528 may extract aspects from the enriched provider data 520 and provide them to one or more third parties.

FIGS. 5C and 5D show an exemplary data model 550 for maintaining and updating provider databases for recommending providers to users in accordance with one or more examples of the present application. For example, FIGS. 5C and 5D show a data model 550 that may be used by the overview 500 and/or the PMRCS 108 to perform one or more functionalities described herein. For instance, the data model 550 may be based on the HL7 FHIR standards described above and includes a plurality of FHIR-based elements such as the Contact Person, Telecom, Address, Identifier, Practitioner, Referral Method, Type, Category, Healthcare Services, Specialty, Period, Organization, and Organization Affiliation. These may be described in further detail above. In addition, the data model 550 includes PMRCS enrichment data (e.g., the enriched provider data 520 described above) such as Provider Scoring, which may include Behavior Score, Patient Review Score, Scheduling Score, Time Spent on Patient Score, Wait Time Score, and/or model and/or vectors (e.g., for AI recommendations). The data model 550 may further include auxiliary data such as Patient Review, which may include Patient Identifier, Geolocation, Review Text, and Score.

The Organization may be associated with a grouping of people or organizations with a common purpose (e.g., a group of providers). The Practitioner may be associated with a person who is directly or indirectly involved in the provisioning of healthcare of related services (e.g., physicians, dentists, pharmacists, and so on). The Healthcare Service may be associated with a category of services that are provided by an organization at a location (e.g., pharmacy and/or psychology services). The Organization Affiliation may be associated with a relationship between two distinct organizations.

The data model 550 might not be strictly an FHIR data model but instead an FHIR-inspired model, which illustrates the relationship between the core data and the enrichment data. The data model 550 shows the relationships in an entity relationship diagram (ERD) Crow's Foot format. In addition, FIG. 5C shows a first portion of the data model 550 and FIG. 5D shows a second portion of the data model 550. For example, the Practitioner, Healthcare Services, and Organization Affiliation blocks are repeated in FIGS. 5C and 5D. These blocks in FIG. 5C are shown to connect to additional blocks in FIG. 5D.

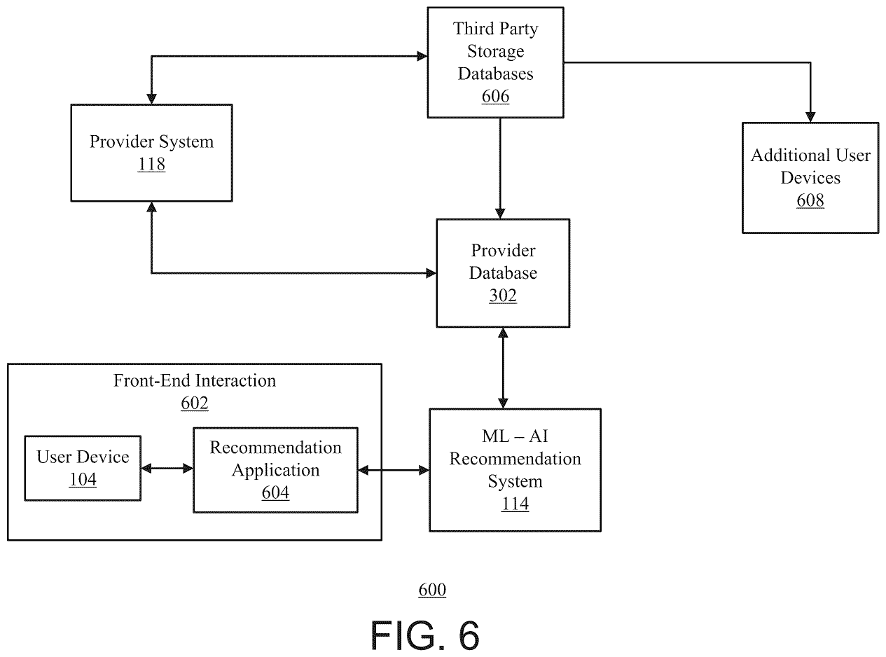

FIG. 6 is an exemplary participant environment 600 for maintaining and updating provider databases for recommending providers to user in accordance with one or more examples of the present application. For instance, the participant environment 600 includes front-end interactions 602, which include a user device 104 and a recommendation application 604. The user device 104 and the recommendation application 604 are described above. Furthermore, the ML-AI recommendation system 114 may obtain information from the provider database 302, and generate and provide a list of recommended providers to the user device 104.

The provider database 302 may further be in communication with third party storage databases 606 (e.g., databases that provide the third party provider review data 524, the third party provider data 518, and/or other third party data), a clinical provider system 118, and additional user devices 608.

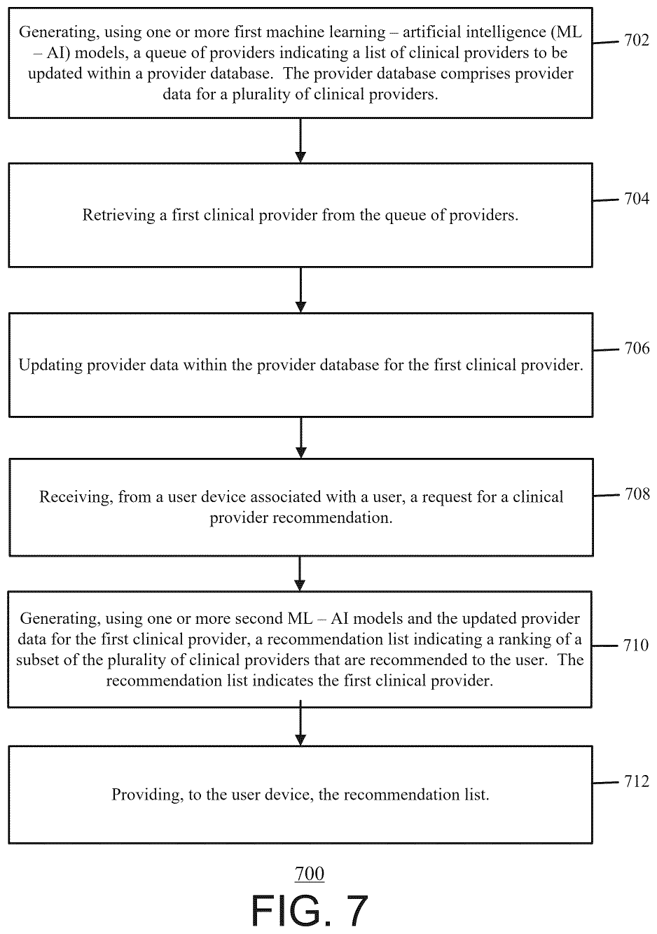

FIG. 7 is an exemplary process 700 for maintaining and updating provider databases and providing recommended providers to users in accordance with one or more examples of the present application. The process 700 may be performed by one or more entities of environment 100 shown in FIG. 1 such as the PMRCS 108. It will be recognized that any of the following blocks may be performed in any suitable order, and that the process 700 may be performed in any suitable environment. The descriptions, illustrations, and processes of FIG. 7 are merely exemplary and the process 700 may use other descriptions, illustrations, and processes for maintaining and updating provider databases and providing recommended providers to users.

At block 702, the PMRCS 108 populates, using one or more first machine learning-artificial intelligence (ML-AI) models, a queue of providers 408 indicating a list of clinical providers to be updated within a provider database 302. The provider database 302 comprises provider data for a plurality of clinical providers. At block 704, the PMRCS 108 retrieves a first clinical provider from the queue of providers 408. At block 706, the PMRCS 108 updates provider data within the provider database for the first clinical provider. At block 708, the PMRCS 108 receives, from a user device 104 associated with a user 102, a request for a clinical provider recommendation. At block 710, the PMRCS 108 generates, using one or more second ML-AI models and the updated provider data for the first clinical provider, a recommendation list indicating a ranking of a subset of the plurality of clinical providers that are recommended to the user. The recommendation list indicates the first clinical provider. At block 712, the PMRCS 108 provides, to the user device 104, the recommendation list. The blocks 702-712 are described in further detail above.

In some examples, the PMRCS 108 may generate and/or use vectorized data representations to perform one or more tasks and/or functions such as generating and providing the list of recommended providers and/or updating the provider database 302. To perform the two functionalities (e.g., generate the list of recommended providers and update the provider database 302), the PMRCS 108 may use a unified model path (e.g., a single model or set of models to perform both functionalities) or a distinct model path (e.g., two separate models or two separate sets of models to perform the two functionalities).

For example, in some variations, the systems 110-116 of the PMRCS 108 may use a unified model path that uses a single model or a single set of models to perform provider quality analysis (e.g., generating the list of recommended providers for a user 102) and data quality analysis (e.g., updating the provider database 302) . . . . In other words, the ML-AI recommendation system 110 and the provider assessment system 114 may generate the list of recommended providers and update the provider database 302 based on using the same model or set of models (e.g., the same encoder or set of encoders). For instance, after collecting data from various data sources and/or platforms (e.g., collecting patient reviews from the ML-AI patient review system 112, collecting the provider data including new provider data that is obtained at block 414, and/or collecting other data and information described above that may be used by the PMRCS 108), the PMRCS 108 may store the data in a central location (e.g., a central repository such as the provider database 302 and/or another database). Subsequently, the PMRCS 108 may define a feature space, generate the vectorized data representations within the defined feature space for the data using the ML-AI model, and generate the list of recommended providers and/or update the provider database 302 based on a comparison (e.g., based on performing cosine similarity). In some instances, for the unified model path, to perform the comparison, the PMRCS 108 may use vector proximity comparisons (e.g., cosine similarity) to determine the correlation between vectors and perform one or more selections based on the correlations (e.g., select a list of recommended providers for the user 102 and/or select a provider to include within the queue 408). For instance, based on using the ML-AI model to generate vectorized data representations for determining data that was recently updated, whether there are any complaints about the provider being able to be reached on the phone, recent sale/practice transfer history for the provider, and so on, the PMRCS 108 may perform the selections. This will be described in further detail in FIG. 8.

FIG. 8 is an exemplary process for providing recommended providers to users using vectorized data representations in accordance with one or more examples of the present application. The process 800 may be performed by one or more entities of environment 100 shown in FIG. 1 such as the PMRCS 108. It will be recognized that any of the following blocks may be performed in any suitable order, and that the process 800 may be performed in any suitable environment. The descriptions, illustrations, and processes of FIG. 8 are merely exemplary and the process 800 may use other descriptions, illustrations, and processes for providing recommended providers to users using vectorized data representations. Process 800 will first describe using the unified model path, and then the distinct model path will be described.

At block 802, the PMRCS 108 may collect data and store the data associated with a plurality of clinical providers within one or more databases. For instance, in the unified model path, the PMRCS 108 may store the data within a central location (e.g., a centralized database such as an enterprise provider operational data store 502 that is shown in FIG. 5A). For example, the PMRCS 108 may collect data such as patient review data, provider data, and/or other data and information described above (e.g., the PMRCS 108 may collect data from a plurality of different data sources such as the clinical provider systems, the third party provide data sources such as data sources that provide FHIR data, patient review data sources, and/or other data sources). The collected data may include objective data (e.g., portions of the provider data such as telephone numbers and/or office locations as well as a time duration since their last update) and subjective data (e.g., patient sentiments).

At block 804, the PMRCS 108 may perform data vectorization using one or more ML-AI models to generate vectorized data representations of the collected data. For instance, as a preliminary step, the PMRCS 108 may define a feature space, which may refer to a number of dimensions (e.g., an “n” number of dimensions (n-dimensions)) where the data points associated with the collected data other than the target variable may reside. For instance, the target variable may be a variable associated with the provider quality analysis and/or the data quality analysis (e.g., whether a provider is of “good” quality for the user 102 and/or whether the data for the provider is of “good” quality). In the unified model path, a single target variable may be used for both the data quality analysis and the provider quality analysis. The PMRCS 108 may use the ML-AI model to perform data vectorization that maps the collected data (other than the target variable) into the defined feature space. For instance, to perform data vectorization, the PMRCS 108 may process the collected data using the ML-AI model to generate code (e.g., vectors within the feature space) such that multiple operations may be performed simultaneously on a series of data points. For example, based on the patient sentiments, provider data, and/or other collected data, the PMRCS 108 may generate a vector for each provider (e.g., a data point for each of the providers within the n-dimensional feature space, and the data point may include a plurality of vectorized data representations associated with the collected data).

To perform the data vectorization, the PMRCS 108 may utilize an ML-AI model such as a set of encoders and/or other ML-AI techniques (e.g., a natural language processing (NLP) process, model, and/or technique). For example, using the ML-AI model (e.g., the set of encoders), the PMRCS 108 may convert (e.g., map) the collected data into vectorized data representations within the feature space. For instance, in some examples, each dimension of the n-dimensions of the feature space may be associated with a feature (e.g., a particular type of data such as a telephone number or an office location of a provider). The feature may indicate a feature score, and the score may be based on a time duration since the latest update of the particular type of data. For example, a first provider may have a telephone number (e.g., on their website) that was updated a month ago and a second provider may have a telephone number that was updated a year ago. The PMRCS 108 may obtain, from the centralized location, provider data indicating the time duration of the last update for the first and second providers. Then, the PMRCS 108 may use the ML-AI model (e.g., an encoder from the set of encoders) to generate a feature score for the first provider and the second provider. The feature score for the first provider may be more positive than the feature score for the second provider. For instance, within the dimension of the feature space for the feature, a positive score may indicate “good” (e.g., a more recent update of the telephone number) and a negative score may indicate “bad” (e.g., a less recent update of the telephone number). Given the updates of the telephone number for the first and second providers, the PMRCS 108 may use the ML-AI model to generate a feature score for the first provider that is more positive than a feature score for the second provider. The PMRCS 108 may use the ML-AI model to generate feature scores for additional features for the collected data.

In some examples, the collected data may include subjective data such as patient reviews. To generate feature scores for the subjective data, the PMRCS 108 may first use feature engineering to identify and/or extract key sentiments (e.g., frustrated, happy, indifferent, angry, and/or anxious) from the patient reviews. Subsequently, the PMRCS 108 may process the extracted key sentiments utilizing the ML-AI model to generate feature scores for the providers (e.g., feature scores indicating sentiments for the providers). The processing of the subjective data will be described in further detail below.

After generating the feature scores, the PMRCS 108 may obtain a vector associated with the feature scores (e.g., the vectorized data representations) for the providers. For instance, the vector may indicate a data point within the feature space for the provider, with more similar providers being closer in proximity (e.g., distance) within the feature space.

In other words, the PMRCS 108 may perform data vectorization for each of the plurality of clinical providers. For instance, for a first clinical provider, the PMRCS 108 may generate a plurality of first vectorized data representations for the first clinical provider based on processing first collected data associated with the first clinical provider using the one or more ML-AI models (e.g., processing provider data and/or patient reviews for the first clinical provider using the ML-AI models). After generating the first vectorized data representations, the PMRCS 108 may generate a first clinical provider vector for the first clinical provider, and map the first clinical provider vector into the feature space. In some instances, to process the first collected data, the PMRCS 108 may separate the collected data into subsets of data (e.g., into telephone data, office location data, and so on). Afterwards, the PMRCS 108 may use an encoder from the ML-AI models to generate a vectorized data representation for each subset of data (e.g., a vectorized data representation for the telephone number of the first clinical provider). In some instances, the subsets of data may indicate a time duration since a last update of a data attribute (e.g., telephone data) for the first clinical provider, and the first vectorized data representation may be based on the time duration (e.g., if the provider has updated their telephone number within the last month, the first vectorized data representation may be more positive than if the provider has updated their telephone number within the last year).

At block 806, based on performing data vectorization and obtaining a feature space for the providers, the PMRCS 108 may perform one or more functionalities (e.g., the data quality analysis and/or the provider quality analysis). For example, the PMRCS 108 may compare resulting vectors using one or more algorithms, processes, and/or techniques to perform the functionalities. For instance, the PMRCS 108 may be able to compare the resulting vectors using cosine similarity, which may be the dot product of two vectors (e.g., a vector A for a first provider and a vector B for a second provider) divided by the product of their magnitudes. A value closer to 1 indicates greater proximity (e.g., similarity), while a value closer to −1 indicates greater dissimilarity. The expression for cosine similarity is shown below:

$$cosine similiarity = \frac{Vector A \cdot Vector B}{‖ Vector A ‖ ‖ Vector B ‖}$$

In other words, to perform provider quality analysis (e.g., generating the list of recommended providers for a user 102), the PMRCS 108 (e.g., the ML-AI recommendation system 110) may obtain, from a user device 104, a request for a clinical provider recommendation, and the request may comprise user information associated with a user 102. The PMRCS 108 may process the user information using the one or more ML-AI models to generate a user vector, and compare the user vector with the vectorized data representations using cosine similarity to determine a subset of clinical providers to recommend to the user 102. Then, the PMRCS 108 may generate a list of recommended providers based on the subset of clinical providers and provide the list back to the user device 104.

For instance, the PMRCS 108 may use the ML-AI model to generate a vector associated with the user information from the user 102. For example, as mentioned above, the user 102 may seek a recommendation for a new provider and may provide a request comprising user information (e.g., user characteristics and/or provider characteristics). The user device 104 may obtain the user characteristics and the provider characteristics from the user 102, and provide the user characteristics and the provider characteristic to the PMRCS 108. The PMRCS 108 may generate a vector using the ML-AI model based on the user characteristics and the provider characteristics (e.g., the PMRCS 108 may perform data vectorization on the user's circumstance such as the user's demographics and/or the user's particular problem). For instance, the PMRCS 108 may process the user information (e.g., the user characteristics and/or the provider characteristics) to generate a user vector. Then, the PMRCS 108 may compare the generated vector for the user 102 with the vectors for the providers within the feature space. For instance, the PMRCS 108 may use cosine similarity to compare the vectors and obtain the closest vectors in the n-dimensional feature space. The PMRCS 108 may generate a list of recommended providers for the user 102 based on the comparison and one or more thresholds (e.g., a distance threshold or a number threshold). For example, the PMRCS 108 may seek to provide a list of five recommended providers (e.g., the number threshold is five), and determine (e.g., using cosine similarity) the five closest provider vectors to the user generated vector. In other words, the number threshold may indicate a number of clinical providers to provide back to the user device 104. The PMRCS 108 may generate the list of recommended providers based on the determination, and provide the list of recommended providers back to the user device 104.

To perform data quality analysis, the PMRCS 108 (e.g., the provider assessment system 114) may determine an ideal vector indicating ideal high quality data for an ideal clinical provider, and measure vector proximity based on the ideal vector. For instance, the PMRCS 108 may generate an ideal vector that represents gold standard provider data (e.g., provider data with recent updates and/or other factors). The PMRCS 108 may perform vector proximity (e.g., cosine similarity) to compare the ideal vector with vectors for the providers, and populate a queue of providers 408 to be updated based on the comparison. For example, referring to FIG. 4 and block 404, the provider assessment system 114 may process the provider data records by comparing the ideal vector with a vector associated with the provider from the retrieved provider data records. Then, at block 406, based on the comparison (e.g., a cosine similarity value that is between 1 and −1), the provider assessment system 114 may place the provider on the validation queue 408. For instance, as mentioned above, based on a low value (e.g., a value closer to −1 indicating a great dissimilarity between the ideal vector and the vector for the provider), the provider assessment system 114 may place the provider at a higher position (e.g., a top position) of the queue 408.

In other words, for a provider, the PMRCS 108 may obtain data quality features (e.g., recency of the update, accuracy of the data, and so on) associated with the provider data as well as provider quality features (e.g., wait times, patient reviews, bedside manner, and so on) associated with the provider data. When generating the list of recommended providers, the PMRCS 108 may utilize the data quality features of the provider to determine the placement of the provider within the validation queue 408. Similarly, the ideal vector may indicate ideal data quality features (e.g., data quality features indicating that the provider data is recent, validated, verified, and/or authoritative data provenance). The PMRCS 108 may utilize the ideal vector indicating the ideal data quality features to place the provider within the validation queue 408. To put it another way, to determine the data quality score for the provider, which is then used to place the provider into the validation queue 408, a cosine similarity score that is based on a cosine similarity expression (e.g., the cosine similarity expression shown above) may be used. Vector A of the cosine similarity expression may be the provider vector (e.g., the data quality features associated with the provider data) and Vector B of the cosine similarity expression may be the ideal vector (e.g., an ideal vector indicating the ideal data quality features).

Additionally, and/or alternatively, at block 414, the provider update system 116 may update the provider data such as by updating one or more vectorized data representations (e.g., feature scores) associated with the provider based on the new provider data. For example, based on obtaining new provider data, the provider update system 116 may use the ML-AI model to generate new feature scores for the provider. Subsequently, the provider update system 116 may replace the previous feature scores from the provider database 302 with the new feature scores. Therefore, the provider update system 116 may generate a new vector within the feature space for the provider based on the new provider data.

In other words, the PMRCS 108 may generate and store a plurality of vectors for the plurality of clinical providers within the provider database 302. Subsequently, the PMRCS 108 may obtain an ideal vector for updating the provider database 302, populate a queue for updating the provider database 302 based on comparing the ideal vector to the vectors from the provider database 302, and update the provider database 302 based on the queue. For instance, using block 412, the PMRCS 108 may obtain new provider data and then at block 414, the PMRCS 108 may generate new vectorized data representations based on the new provider data and update a vector within the provider database based on the new vectorized data representations.

In some examples, to process the subjective data such as the patient reviews, the PMRCS 108 may use feature engineering. For example, the PMRCS 108 may use feature engineering to extract key sentiments from the patient reviews (e.g., happy, anxious, frustrated, and so on). The PMRCS 108 may then generate vectorized data representations for the extracted key sentiments (e.g., the sentiment analysis from the patient reviews) using one or more algorithms, models, and/or techniques such as using the ML-AI model and/or cluster analysis. For example, the PMRCS 108 may use cluster analysis, which may allow for good coverage for important sentiments, to analyze the extracted key sentiments and generate the vectorized data representations. Afterwards, the PMRCS 108 may combine the generated vectorized data representations for the extracted key sentiments with the feature scores for the objective data to generate the vectors for the providers that are within the feature space.

In some instances, the PMRCS 108 may generate text embeddings for the sentiment analysis, the patient reviews, and/or generative artificial intelligence (AI) interface interactions with patients. For instance, for sentiment analysis and patient reviews, the PMRCS 108 may first generate text embeddings for the extracted key sentiments (e.g., based on using an NLP model and/or technique). Then, the PMRCS 108 may generate the vectorized data representations for the extracted key sentiments based on the generated text embeddings (e.g., by using cluster analysis). Additionally, and/or alternatively, the PMRCS 108 may obtain structured and/or unstructured patient interactions. For instance, in the unstructured interface, the patient may be able to use free form chat to communicate their interactions (e.g., based on using generative artificial intelligence (AI)). In the structured interface, patients may be able to select/unselect checkboxes and/or use filters to describe their needs and preferences for a provider. The PMRCS 108 may obtain the structured and/or unstructured patient interactions, generate text embeddings from the structured and/or unstructured patient interactions, and generate vectorized data representations for structured and/or unstructured patient interactions based on the text embeddings.

In some examples, to train the one or more models described above (e.g., the ML-AI model), the PMRCS 108 may perform supervised and/or unsupervised training. For instance, the PMRCS 108 may obtain training data (e.g., data quality training data and/or provider quality training data). For supervised training, the PMRCS 108 may label a set of data from the training data, and use the labelled data to train the ML-AI model and/or other models. Additionally, and/or alternatively, the PMRCS 108 may perform unsupervised training. For instance, the PMRCS 108 may perform feature and/or cluster detection on the training data (e.g., the raw set of data), and may then align this with the sentiment analysis of patient reviews. In other words, the PMRCS 108 may seek to isolate the data features as compared to the provider features, and attempt to match up each feature type with the sentiment.

In some variations, the PMRCS 108 may use a distinct model path to perform the two functionalities. For example, the PMRCS 108 may use two separate ML-AI models and/or two separate feature spaces to generate the list of recommended providers and to update the provider database 302. For example, the PMRCS 108 may use a first ML-AI model to score each provider record (e.g., provider data) and train the first ML-AI model to provide a weighted average. Then, the PMRCS 108 may use a second ML-AI model to generate the list of recommended providers. With distinct ML-AI models, the PMRCS 108 may perform feature discovery and attempt to align reviews that are similar in sentiment.

In other words, within the distinct model path, the PMRCS 108 may define two feature spaces—a first feature space to update the provider database 302 and a second feature space to generate the list of recommended providers. Based on the collected data and for each provider, the PMRCS 108 may use the collected information to generate a first vector for the first feature space and a second vector for the second feature space. For instance, the first ML-AI model may map the first vector for the clinical provider to the first feature space and the second ML-AI model may map the second vector for the clinical provider to the second feature space. For example, based on a subset of the collected information, the PMRCS 108 may generate a first vector using the first ML-AI model that is used to update the provider database 302 (e.g., a first vector indicating a data quality score for the provider). Then, based on another subset of the collected information and/or based on the first vector, the PMRCS 108 may generate a second vector using the second ML-AI model that indicates a provider vector for use in generating the list of recommended providers. Following, the PMRCS 108 may generate an ideal vector and compare the ideal vector to provider vectors (e.g., the first vector indicating the data quality score) for the first feature space. Based on the comparison, the PMRCS 108 may populate the queue 408 for updating the provider data within the provider database 302. Further, the PMRCS 108 may generate a user vector for the user 102, and compare the user vector with the provider vectors within the second feature space (e.g., the second vector). Based on the comparison, the PMRCS 108 may generate a list of recommended providers and provide the list to the user device 104.

Additionally, and/or alternatively, based on using the distinct model path, the vectorized data representations for the sentiment analysis and/or the patient reviews may be used differently by the PMRCS 108. For example, the PMRCS 108 may extract the sentiments from the patient reviews and generate vectorized data representations using the first ML-AI model (e.g., the ML-AI model that scores the provider data). Given the two feature spaces, the PMRCS 108 may use the extracted sentiments for the first ML-AI model and/or the second ML-AI model. For example, the PMRCS 108 may process the vectorized data representations for the extracted sentiments along with other collected information using the first ML-AI model to generate the first vector indicating the data quality score for the provider. Additionally, and/or alternatively, using the second ML-AI model, the PMRCS 108 may process the vectorized data representations for the extracted sentiments, the collected information, and/or the first vector to generate the second vector that is used to generate the list of recommended providers.

A number of implementations have been described. Nevertheless, it will be understood that additional modifications may be made without departing from the scope of the inventive concepts described herein, and, accordingly, other examples are within the scope of the following claims. For example, it will be appreciated that the examples of the application described herein are merely exemplary. Variations of these examples may become apparent to those of ordinary skill in the art upon reading the foregoing description. The inventor expects skilled artisans to employ such variations as appropriate, and the inventor intends for the application to be practiced otherwise than as specifically described herein. Accordingly, this application includes all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

It will further be appreciated by those of skill in the art that the execution of the various machine-implemented processes and steps described herein may occur via the computerized execution of processor-executable instructions stored on a non-transitory computer-readable medium, e.g., random access memory (RAM), read-only memory (ROM), programmable read-only memory (PROM), volatile, nonvolatile, or other electronic memory mechanism. Thus, for example, the operations described herein as being performed by computing devices and/or components thereof may be carried out by according to processor-executable instructions and/or installed applications corresponding to software, firmware, and/or computer hardware.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the application and does not pose a limitation on the scope of the application unless otherwise claimed. No language in the specification should be construed as indicating any non-claimed element as essential to the practice of the application.

1. A method, comprising:
obtaining training data for training a first machine learning-artificial intelligence (ML AI) model and a second ML-AI model;
training, using the training data, the first ML-AI model to operate in a first feature space of a plurality of feature spaces;
training, using the training data and outputs from the first ML-AI model, the second ML-AI model to operate in a second feature space of the plurality of feature spaces;
collecting data for a plurality of clinical providers from a plurality of different data sources;
performing data vectorization for the plurality of clinical providers using the first ML-AI model and the second ML-AI model to map the collected data into the plurality of feature spaces, wherein the first ML-AI model maps the collected data into a first vectorized data representations in the first feature space and the second ML-AI model maps the collected data into a second vectorized data representations in the second feature space;
obtaining, from a user device, a request for a clinical provider recommendation, wherein the request comprises user information associated with the user;
processing the user information using the one or more ML-AI models to generate a user vector that is in the second feature space;
comparing the user vector with the second vectorized data representations to determine a subset of the plurality of clinical providers to recommend to the user;
generating a list of recommended providers that is responsive to the request from the user device based on the subset of the plurality of clinical providers; and
providing, to the user device, the list of recommended providers.

2. The method of claim 1, wherein collecting the data for the plurality of clinical providers comprises:
obtaining provider data from a plurality of clinical provider systems; and
obtaining patient review data from one or more patient review data sources.

3. The method of claim 1, wherein performing the data vectorization for the plurality of clinical providers comprises:
generating a plurality of first clinical provider vectorized data representations for a first clinical provider based on processing first collected data, from the collected data, associated with the first clinical provider using the first ML-AI model; and
mapping a first clinical provider vector for the first clinical provider into the first feature space based on generating the first clinical provider vector using the plurality of first clinical provider vectorized data representations.

4. The method of claim 3, wherein the first ML-AI model comprises a set of encoders, and wherein generating the plurality of first clinical provider vectorized data representations comprises:
separating the first collected data associated with the first clinical provider into a plurality of subsets of the first collected data; and
processing each of the plurality of subsets of the first collected data using an encoder from the set of encoders to generate a first clinical provider vectorized data representation from the plurality of first clinical provider vectorized data representations.

5. The method of claim 4, wherein a first subset of the plurality of subsets of the first collected data indicates a time duration since a last update of a data attribute for the first clinical provider, and wherein the first clinical provider vectorized data representation is based on the time duration since the last update of the data attribute for the first clinical provider.

6. The method of claim 1, wherein the user information comprises user characteristics associated with the user and provider characteristics associated with desired clinical providers.

7. The method of claim 1, wherein comparing the user vector with the second vectorized data representations to determine the subset of the plurality of clinical providers to recommend to the user is based on using cosine similarity and a number threshold indicating a number of clinical providers that are within the subset of the plurality of clinical providers.

8. The method of claim 1, wherein performing the data vectorization for the plurality of clinical providers comprises:
generating a plurality of vectors for the plurality of clinical providers within the second feature space, wherein each of the plurality of vectors is associated with a clinical provider from the plurality of clinical providers and comprises one or more of the first vectorized data representations or the second vectorized data representations; and
storing the plurality of vectors within a provider database, wherein determining the subset of the plurality of clinical providers to recommend to the user comprises comparing the user vector with the plurality of vectors stored within the provider database using cosine similarity.

9. The method of claim 8, further comprising:
obtaining an ideal vector for updating the provider database;
populating a queue for updating the provider database based on comparing the ideal vector to one or more vectors, of the plurality of vectors, from the provider database; and
updating the provider database based on the queue.

10. The method of claim 9, wherein updating the provider database based on the queue comprises:
determining a first clinical provider from the queue;
obtaining new provider data for the first clinical provider based on using a voice artificial intelligence (AI) agent, one or more application programming interface (API) calls to a provider system associated with the first clinical provider, or performing web scraping;
generating new vectorized data representations for the first clinical provider based on the new provider data; and
updating a first vector for the first clinical provider within the provider database based on the new vectorized data representations.

11. The method of claim 1, wherein the collected data comprises patient review data and provider data, and wherein perform the data vectorization for the plurality of clinical providers comprises:
using feature engineering to extract sentiments from the patient review data;
generating the first vectorized data representations within the first feature space based on using the first ML-AI model to process the provider data; and
generating the second vectorized data representations within the second feature space based on the extracted sentiments from the patient review data.

12. The method of claim 11, wherein generating the second vectorized data representations within the second feature space comprises:
generating text embeddings for the extracted sentiments; and
performing cluster analysis for the text embeddings to generate the second vectorized data representations.

13. (canceled)

14. A system, comprising:
one or more processors; and
a non-transitory computer-readable medium having processor-executable instructions stored thereon, wherein the processor-executable instructions, when executed by the one or more processors, facilitate:
obtaining training data for training a first machine learning-artificial intelligence (ML-AI) model and a second ML-AI model;
training, using the training data, the first ML-AI model to operate in a first feature space of a plurality of feature spaces;
training, using the training data and outputs from the first ML-AI model, the second ML-AI model to operate in a second feature space of the plurality of feature spaces;
collecting data for a plurality of clinical providers from a plurality of different data sources;
performing data vectorization for the plurality of clinical providers using the first ML-AI model and the second ML-AI model to map the collected data into the plurality of feature spaces, wherein the first ML-AI model maps the collected data into a first vectorized data representations in the first feature space and the second ML-AI model maps the collected data into a second vectorized data representations in the second feature space;
obtaining, from a user device, a request for a clinical provider recommendation, wherein the request comprises user information associated with the user;
processing the user information using the one or more ML-AI models to generate a user vector that is in the second feature space;
comparing the user vector with the second vectorized data representations to determine a subset of the plurality of clinical providers to recommend to the user;
generating a list of recommended providers that is responsive to the request from the user device based on the subset of the plurality of clinical providers; and
providing, to the user device, the list of recommended providers.

15. The system of claim 14, wherein collecting the data for the plurality of clinical providers comprises:
obtaining provider data from a plurality of clinical provider systems; and
obtaining patient review data from one or more patient review data sources.

16. The system of claim 14, wherein performing the data vectorization for the plurality of clinical providers comprises:
generating a plurality of first clinical provider vectorized data representations for a first clinical provider based on processing first collected data, from the collected data, associated with the first clinical provider using the first ML-AI model; and
mapping a first clinical provider vector for the first clinical provider into the first feature space based on generating the first clinical provider vector using the plurality of first clinical provider vectorized data representations.

17. The system of claim 16, wherein the first ML-AI model comprises a set of encoders, and wherein generating the plurality of first clinical provider vectorized data representations comprises:
separating the first collected data associated with the first clinical provider into a plurality of subsets of the first collected data; and
processing each of the plurality of subsets of the first collected data using an encoder from the set of encoders to generate a first clinical provider vectorized data representation from the plurality of first clinical provider vectorized data representations.

18. The system of claim 17, wherein a first subset of the plurality of subsets of the first collected data indicates a time duration since a last update of a data attribute for the first clinical provider, and wherein the first clinical provider vectorized data representation is based on the time duration since the last update of the data attribute for the first clinical provider.

19. The system of claim 14, wherein the user information comprises user characteristics associated with the user and provider characteristics associated with desired clinical providers.

20. A non-transitory computer-readable medium having processor-executable instructions stored thereon, wherein the processor-executable instructions, when executed, facilitate:
obtaining training data for training a first machine learning-artificial intelligence (ML-AI) model and a second ML-AI model;
training, using the training data, the first ML-AI model to operate in a first feature space of a plurality of feature spaces;
training, using the training data and outputs from the first ML-AI model, the second ML-AI model to operate in a second feature space of the plurality of feature spaces;
collecting data for a plurality of clinical providers from a plurality of different data sources;
performing data vectorization for the plurality of clinical providers using the first ML-AI model and the second ML-AI model to map the collected data into the plurality of feature spaces, wherein the first ML-AI model maps the collected data into a first vectorized data representations in the first feature space and the second ML-AI model maps the collected data into a second vectorized data representations in the second feature space;
obtaining, from a user device, a request for a clinical provider recommendation, wherein the request comprises user information associated with the user;
processing the user information using the one or more ML-AI models to generate a user vector that is in the second feature space;
comparing the user vector with the second vectorized data representations to determine a subset of the plurality of clinical providers to recommend to the user;
generating a list of recommended providers that is responsive to the request from the user device based on the subset of the plurality of clinical providers; and
providing, to the user device, the list of recommended providers.

21. The method of claim 1, wherein training the second ML-AI model to operate in the second feature space comprises:
processing training vectorized data representations for extracted sentiments using the first ML-AI model to generate the outputs, wherein the outputs comprise first training vectors; and
training the second ML-AI model using the training vectorized data representations, the first training vectors, and the training data.
